(19) United States
(12) Patent Application Publication
(10) Pub. No.: US 2007/0184430 A1
(43) Pub. Date: Aug. 9, 2007

(54) Method for analysing a tissue sample

(76) Inventors: Axel Niendorf, Hamburg (DE); Klaus Bendrat, Hamburg (DE)

Correspondence Address: COHEN, PONTANI, LIEBERMAN & PAVANE, 551 FIFTH AVENUE, SUITE 1210, NEW YORK, NY, 10176, US

(21) Appl. No.: 10/590,927

(22) PCT Filed: Feb. 25, 2005
(86) PCT No.: PCT/EP05/01984

§ 371 Date: Apr. 9, 2007

(30) Foreign Application Priority Data
Feb. 26, 2004 (DE) 2004 009 934.0

(51) Int. Cl. C12Q 1/00; C12Q 1/68; G01N 1/30

(52) U.S. Cl. 435/4; 435/6; 435/40.5

(57) ABSTRACT

A method of analyzing a patient tissue sample to determine its diseased tissue fraction while essentially preserving the genomic and/or proteomic and/or epigenomic and/or biophysical properties of the tissue sample. The method includes preparing sections from the tissue sample, subjecting at least one of the sections to a histological/cytological examination, and subjecting at least another one of the sections to a non-morphological analytical testing. In the histological/cytological examination, a quantitative fraction of diseased tissue/cells and/or another morphological aspect is determined by an image processing system, and the determined quantitative fraction of diseased tissue/cells and/or another morphological aspect is used as a reference quantity on which evaluation of the result of the non-morphological analytical testing is based.

The present invention concerns a method for analyzing a tissue sample in accordance with the introductory clause of Claim 1.

Many diseases are based on morphologically detectable tissue changes. These diseases include benign and malignant tissue proliferations, cancer, inflammations, and neurodegenerative diseases.

In many cases, it is standard practice to analyze tissue samples by histological methods to diagnose diseases of these types. These methods have become so well established that they provide high diagnostic reliability for many diseases and are therefore thought of as the “gold standard”, e.g., in tumor diagnosis.

In the meantime, however, molecular-biological characterization of diseases is playing an increasingly important role both in basic research and in clinical diagnostics. Diseased tissues differ from healthy tissues in certain quantities that can be detected by molecular-biological means, e.g., genomic sequences, mRNA sequences, the proteome, the methylation of the genomic DNA, the presence of viral or bacterial nucleic acids, and/or the presence of pathogenic molecules.

Some types of diseases are even distinguished by characteristic gene sequence patterns, gene expression profiles, or DNA methylation patterns, which can be detected by molecular-biological methods and used for the diagnosis of these diseases. Many diseases, e.g., variants of various types of tumors, which often differ significantly from one another in their virulence and their susceptibility to treatment, cannot be diagnosed at all without taking this approach, since they cannot be differentiated in the histological section. Molecular-biological this approach, since they cannot be differentiated in the histological section. Molecular-biological characterization is thus a promising approach to the more precise diagnosis of tissue diseases and may possibly allow individualized therapies. The recently developed methods of molecular biology known as high-throughput analysis based on nucleic acid or protein arrays (“microarrays”) are especially suitable for this purpose.

The advantages of a molecular approach in tissue diagnostics are outlined in the paper “Microarray and histopathological analysis of tumors: The future and the past?” by S. R. Lahkani and A. Ashworth (Nature Reviews 2001, 1, 51).

An important problem with the molecular-biological analysis of a tissue sample is that the results which are obtained are difficult to interpret. It is conceivable, for example, that in a known tumor X a certain gene Y is typically overexpressed. A pure tumor sample would produce an immediately apparent result in the molecular-biological analysis due to its greatly elevated gene expression value. However, if the analyzed sample contains only a small fraction of tumor tissue, the gene expression value of the overall sample would be only slightly elevated, and it would be difficult to interpret such a gene expression value correctly.

It is also conceivable for a known tumor X′ to have a certain gene expression profile Y′, i.e., a characteristic pattern of gene expression values of two or more genes. In this case, the contamination of the analyzed sample by nontumor tissue would distort the gene expression profile Y′ and thus make a diagnosis impossible.

The paper “A Gene-Expression Signature as a Predictor of Survival in Breast Cancer” by de Vijver et al. (The New England Journal of Medicine 2002, Vol. 347, 1999) describes a method for analyzing a tissue sample for diseased tissue fractions, in which sections are prepared from the tissue sample, at least one of which is subjected to a histological analysis, while at least one other is subjected to nonmorphological analytical testing, namely, a molecular-biological microarray analysis.

In this method, a frozen tissue sample is placed in a microtome, in which a series of sections is prepared. One or more sections of the series are stained with hematoxylin and eosin and histologically evaluated. The fraction of tumor cells in the individual sections is determined. If the fraction is greater than 50%, then corresponding sections from the same series of sections are used for the molecular-biological analysis. If the fraction of tumor cells in the section is less than 50%, then the entire series of sections is discarded.

The knowledge about the composition of the sample that is gained from the preliminary histological examination is thus used only to decide whether the sample does or does not satisfy a certain exclusion criterion.

The aforementioned method is used in basic research, for example, for developing gene expression data banks, and to do this, it reverts to tissue banks with relatively large numbers of tissue samples. If the sample to be analyzed does not satisfy the exclusion criterion, a new sample is taken from the tissue bank, histologically reexamined for its fraction of tumor cells, and then, if necessary, subjected to molecular-biological analysis.

In clinical diagnostics, on the other hand, where often only a small amount of sample material of an individual patient is available, and a quick analysis result is generally needed—especially in the case of intraoperative and perioperative diagnostics—this procedure is impracticable. Specifically, if it were found during the preliminary histological examination of a patient-specific sample that the fraction of tumor cells is too small, and thus the sample does not satisfy the exclusion criterion, it would be necessary to dispense with the molecular-biological analysis altogether, since the available sample material would already have been used and/or no more time would be available to take and analyze a new sample. In a clinical-diagnostic setting, the method described above thus would not allow a molecular-biological analysis of all tissue samples in addition to the histological evaluation.

The objective of the present invention is to make available a diagnostically suitable method for analyzing a tissue sample for diseased tissue fractions and/or other relevant components and their relation to one another, in which individual samples are subjected to a histological and a nonmorphological analytical evaluation.

This objective is achieved with the features of Claims 1 and 2.

These features provide a method for analyzing a tissue sample, whose genomic and/or proteomic and/or epigenomic and/or biophysical properties are essentially preserved, to determine its diseased tissue fractions. As in previously known methods, sections are prepared in the usual way from the tissue sample, and at least one of these sections is subjected to a histological/cytological examination, while at least one other section is subjected to nonmorphological analytical testing.

In the discussion which follows, the term “nonmorphological analytical testing” is understood to mean especially molecular-biological analyses. In this regard, mainly the term “molecular-biological analyses” will be used. In the context of the present invention, however, nonmorphological analytical tests also include biophysical analyses, such as methods for measuring the redox potential, the pH, the temperature, and the oxygen partial pressure or methods for determining metabolites, such as lactate or pyruvate.

In accordance with the invention, at least the quantitative amount of the diseased tissue or diseased cells and/or some other morphological aspect is determined in the tissue sample during the histological/cytological examination by means of an image processing system and then used as a reference quantity on which the evaluation of the results of the nonmorphological analytical testing is based. During the histological/cytological examination, it is also possible to determine, for example, the fraction of necrotic tissue, inflammatory cells, or nonpathological connective tissue, and to take these findings into consideration in the evaluation of the results of the nonmorphological analysis.

The quantitative determination of the fraction of diseased tissue or diseased cells can be made, for example, by first staining a section and then evaluating it by an image processing system, especially a computer-assisted image processing system. A system of this type usually consists of an optical system (e.g., microscope), an image acquisition system (e.g., CCD camera and image acquisition card), a computer, and suitable software. A large number of quantities can be quickly, automatically, and reproducibly determined from histological preparations with a system of this type.

In accordance with the invention, the image acquisition system can be used to quantitatively determine at least the fraction of tumor tissue and/or some other morphological aspect in the tissue sample by means of the image processing system, which is reliably possible with suitable histological pretreatment of the sample, e.g., by counting stained cells and cell nuclei or integration of stained tissue regions (to mention only two examples).

It is thus possible to determine, for example, the reference quantity with which an experimentally determined gene expression pattern can be corrected in order to compare it with the patterns stored in corresponding data banks.

In the evaluation, the fraction of diseased tissue in the sample is quantitatively determined, for example, as stated above, by counting the cell nuclei, and recorded as the reference quantity, e.g., in the form of a percentage. In the molecular-biological analysis of another section, the expression pattern of 10 disease-specific genes, for example, is then quantitatively determined.

To interpret the determined expression pattern, it must be compared with a data bank, in which well-known, standardized expression patterns of a large number of diseases are stored. Standard gene expression profiles of this type are presently being determined in various laboratories around the world for a large number of tumor and tissue types. The method of the invention can make a contribution to this effort, as will be described below.

However, standardized expression patterns cannot be directly compared with the experimentally determined expression pattern, since they refer to standardized samples, which, for example, contain exclusively diseased tissue.

The experimentally determined expression pattern can now be corrected with the previously determined reference quantity and can then be directly compared with the patterns stored in the data bank. Consequently, even the gene expression patterns of samples that have a high proportion of nondiseased tissue can be evaluated in a meaningful way and diagnostically interpreted.

In another variant of the method of the invention, a sample is taken from a tissue sample. At least one portion of this sample is subjected to a histological/cytological evaluation, and at least one other portion of the sample is subjected to nonmorphological analytical testing. It can be just as effective to take several samples from the tissue sample, at least one of which is subjected to a histological/cytological evaluation, while at least one other of the samples is subjected to nonmorphological analytical testing. In this embodiment, it is also possible to use only portions of the individual samples of the tissue sample for the various tests.

In this embodiment, the quantitative fraction of diseased tissue or diseased cells is likewise determined in the tissue sample or the sample during the histological/cytological evaluation, and the quantitative fraction that is determined is then used as a reference quantity on which the evaluation of the results of the molecular-biological testing is based. This variant thus differs from the previous variant only in that one or more samples are prepared instead of sections. Accordingly, the statements made about the previous variant also apply to the samples or their portions.

In another preferred embodiment of the method, the sample is taken from a tissue sample taken from a patient. In this method for an ex vivo sample, the samples or the portions of samples can be taken, for example, by a core sampler or by aspiration with a fine needle. This has the advantage that the tissue sample is not destroyed, and, for example, the tumor margins remain unaffected.

It is also possible to obtain the ex vivo sample or the portions of the sample by the so-called scrape sampling technique. This technique is referred to as “epithelial aggregate separation and isolation”. In this technique, for example, a slide tilted on edge is passed with slight pressure over the cut surface of a tissue sample that has been removed and cut through. In the case of solid tissues, the blade of a scalpel or other suitable instrument can also be passed over the surface. In this process, tumor cell aggregates, which, in contrast to the surrounding nonmalignant tissue, are organized in islands and protrude slightly from the cut surface, are purposefully accumulated on the slide or the blade of the scalpel.

The scrape preparation obtained in this way can be divided directly into two sample portions, one of which, in accordance with the invention, can be used for the histological/cytological examination, while the other can be used for the molecular-biological testing. However, it is also possible first to prepare a suspension from the scrape material thus obtained and then to divide the suspension into two sample portions. The latter method ensures better randomization of the composition of the individual sample portions.

However, it is also possible first to wash and centrifuge the scrape material, to resuspend the resulting pellet, and then to centrifuge again in a density gradient. At least one of the portions obtained in this way is then divided, and, in accordance with the invention, one sample portion is used for the histological/cytological evaluation, and the other is used for the nonmorphological analytical testing. However, the scrape material can also be worked up by any other suitable enriching and/or fractionating technique.

The nonmalignant tissue treated by the scrape technique remains on the cut surface with its tissue structure largely preserved, while the areas in which the tumor cell aggregates had previously settled are absent and thus appear as depressions. The tissue sample can then be cut, e.g., in the conventional way with a microtome. In this regard, the first section of the series of sections corresponds to the cut surface of the tissue sample, which has been depleted by the scrape preparation. This section can be examined, e.g., histologically, for remaining tumor cell aggregates and thus provides information about the success of the sampling. The section can thus serve as a negative control for the evaluation of the test results of the scrape sample.

Further sections of the series of sections, on the other hand, consist of both nonmalignant tissue and tumor tissue and therefore, as described above, can be subjected, in accordance with the invention, to a histological/cytological examination, on the one hand, and to nonmorphological analytical testing, on the other hand. In this regard, it is especially promising to compare the histological/cytological and molecular-biological results that are obtained with those of the scrape preparation.

In all of the methods which have been described, it is always essential that at least two sections or samples (e.g., tissue core samples, fine-needle aspirates, scrape preparations) or at least two portions of a sample are prepared and subjected to histological/cytological and nonmorphological analytical testing.

In addition, it is always essential that at least the quantitative fraction of the diseased tissue or diseased cells in the tissue sample or the sample is determined in the histological/cytological examination and then used as a reference quantity on which the evaluation of the results of the nonmorphological analytical testing is based. The individual sections, samples, or sample portions will be referred to in the discussion below, where appropriate, under the collective term “divided samples”.

Naturally, it is possible in one method to prepare both sections and, e.g., tissue core samples, from one tissue sample. For example, the sections could then be used for the histological/cytological examination, and the tissue core samples taken from the same sample could be subjected to molecular-biological testing. It is equally possible in one method, as has already been described, to prepare both sections and scrape preparations from one tissue sample. Modifications in which different types of preparation of sections, samples, or portions of these samples are combined with one another are therefore also explicitly covered by the present invention.

An important aspect of the invention is that, in the histological/cytological examination, not only the quantitative fraction of the diseased tissue and/or diseased cells and/or other components in the tissue sample is determined, but also their appearance and/or distribution pattern, which can then likewise be used as the basis for evaluating the results of the nonmorphological analytical testing. In this connection, possible parameters that can be determined by the image acquisition system in addition to the aforementioned cell numbers and areas, are, for example, the distance of stained cells and cell nuclei from one another or from other structures, especially, e.g., blood vessels, and the appearance of individual cells (outer contour), etc., to name only a few examples. Basically, all suitable and morphological parameters can be determined which are suitable for supplementary qualification or quantification of diseased cells or tissues.

For example, an appearance that shows numerous outgrowths of a tumor into the surrounding tissue can be an indication of high malignancy of the tumor. On the other hand, another tumor whose molecular-biological characteristics are similar to those of the first tumor but which has a different appearance may be less malignant. Accordingly, in a case of this kind, the appearance would provide important clues for the interpretation of the results of the molecular-biological analysis. The same is true of the distribution pattern of the diseased tissue or diseased cells in the tissue sample.

In an especially preferred modification of the method of the invention, the sections or samples are prepared directly from the fresh tissue sample. This procedure guarantees that the genomic, proteomic, and/or epigenomic properties of the sample are preserved as well as possible. Sections can be prepared, for example, with a vibratome.

In another especially preferred modification of the method of the invention, the tissue sample is frozen before the sections or samples are prepared. This procedure also guarantees preservation of the genomic, proteomic, and/or epigenomic properties of the sample. The sections can be prepared, for example, with a microtome or cryotome.

If necessary, however, it is also possible to preserve the tissue sample before the preparation of the sections or before the samples are taken and then to embed it in a suitable medium. A medium of this type can be, for example, paraffin or some other suitable embedding agent. In this case, it is possible, for example, to prepare sections in the usual way with a microtome. Here again, the important consideration is that the genomic, proteomic, and/or epigenomic properties of the sample are preserved.

Naturally, it can also be provided, for example, that first a fine-needle aspirate or a scrape sample is taken from a fresh tissue sample and subjected to molecular-biological analysis. The tissue sample could then be frozen, and sections could then be prepared in a cryotome and sent for histological/cytological examination. Modifications in which different types of pretreatment and preservation of the tissue sample are combined with one another are therefore also explicitly covered by the present invention.

In general, it can thus be said that all of the steps described above must be selected in such a way that they cause no damage or as little damage as possible to the nucleic acids (DNA, mRNA), proteins, and/or the epigenomic methylation pattern present in the sample.

An especially preferred modification of the method of the invention is particularly well suited for use in clinical diagnostics. For clinical diagnostic purposes, a tissue sample that has been removed from the patient and is to be used for diagnosis is sent as quickly as possible to the pathology laboratory, where it is divided into two samples. The second sample is fixed and embedded for a thorough histological/cytological evaluation at a later time, while the first sample is immediately mounted on a slide, frozen, and cut in a microtome (“quick section”). Individual sections are then histologically stained and immediately evaluated by a pathologist, who reports his diagnosis to the treating physician. Particularly in intraoperative diagnosis, in which the operating surgeon, depending on the diagnosis reported to him by telephone by the pathologist, must make a decision about further surgical procedures that may be necessary, for example, the excision of axillary lymph nodes that may be additionally necessary after the removal of a breast carcinoma, this method has great importance, and, accordingly, great attention must be paid to optimization of the lapses of time.

In the preferred modification, the tissue sample is removed from a patient, mounted on a slide, frozen, and cut with a microtome, and in addition to the sections that are sent for histological/cytological examination, other sections of the same series of sections are used for the nonmorphological analytical testing. Therefore, the pathologist can additionally base his diagnosis on molecular-biological findings and thus increase the reliability of the diagnosis, a point that is of great importance precisely in intraoperative diagnosis.

A great advantage of this additional step of the method is that it can be seamlessly integrated in the routine diagnostic procedure without loss of time. When a suitable method of molecular-biological analysis is used, such as an array-based mRNA analysis, test results can be obtained in a relatively short time and can possibly even be sent back to the treating physician at the same time as the histological findings. Moreover, the excess material that is obtained anyway in the quick section is profitably utilized in this additional step of the method.

In this regard, in a preferred modification of the method, the tissue sample remains on the slide after the preparation of the sections, so that it is available for the preparation of new sections. When a cryotome and the quick procedure are used, it is guaranteed that the tissue sample remains frozen during the entire process and can be put back in the freezer for storage without being subjected to any significant temperature effects. It can also be provided that the sample increases in temperature before or during the cutting and is then frozen again.

Both procedures create the possibility, for example, of later reexamination of a sample that has already been subjected to histological/cytological examination and/or molecular-biological analysis, so that, for example, methods can be applied that did not exist at the earlier time, or the sample can be reexamined as part of a study. If the tissue sample was preserved in a different way, for example, as described above, by embedding it in a suitable embedding agent, naturally, it can also be provided that it remains on the slide after preparation of the sections and is available for the preparation of new sections. In this case, intermediate freezing becomes unnecessary.

This modification is especially advantageous in conjunction with the modification that will now be described, in which the slide on which the tissue sample is mounted is designed in such a way that it can be reproducibly placed in the microtome, so that the tissue sample has the same relative orientation to the microtome in each preparation of sections. This guarantees that the sample analyzed at a later time is largely the same as the earlier sample, so that the histological/cytological and/or molecular-biological findings can be directly compared with each other.

The consideration of the quantitative fraction of diseased tissue or diseased cells in the tissue sample in the evaluation of the results of the nonmorphological analytical testing is meaningful only if it can be assumed that the sections, the samples, or the portions of the samples that are used for the histological/cytological examination and the nonmorphological analytical testing have essentially the same composition.

Ideally, this would be the case if the same sample could be used for both tests. In the preparation for the histological test, a sample is usually stained and/or fixed, which adversely affects the genomic, proteomic, and/or epigenomic properties of the sample. On the other hand, in the preparation for the molecular-biological test, a sample is generally homogenized, which results in the loss of all topological information of the sample. The preparation of a tissue sample for one test makes the sample unusable for the other test. Therefore, different divided samples must be used for the two tests.

When, for example, the divided samples are two immediately adjacent sections of a microtome section series, it can generally be assumed that these two sections have essentially the same composition. This assumption is also generally accepted by experts in pathology.

However, it is conceivable that the divided samples differ from each other in their composition, i.e., that, for example, in the divided sample that is subjected to histological/cytological examination, the tumor tissue has a different quantitative fraction from the divided sample that is subjected to molecular-biological analysis. This is the case, for example, when the section series catches a region of the tumor margin that runs parallel to the sectional plane. Consequently, a quantitative reference quantity determined from the histologically/cytologically examined divided sample would provide a false tumor tissue fraction in the molecular-biologically analyzed divided sample and thus lead to an erroneous correction of the experimentally determined expression pattern. A similar situation occurs when the tumor tissue has a different appearance or a different distribution pattern in the histologically/cytologically examined divided sample than in the molecular-biologically analyzed divided sample.

Therefore, in an especially preferred embodiment of the method of the invention, at least two sections are used for the histological/cytological examination. These sections are selected in a way that ensures that the section or sections sent for nonmorphological analytical testing were located between these sections in situ. In practice, two sections of a microtome section series that are not immediately adjacent to each other are stained and evaluated histologically/cytologically, while other sections of the microtome section series that are located between these sections are homogenized and subjected, for example, to an array-based mRNA analysis. The number of sections used for this purpose depends in this case, for example, on the amount of mRNA required for the analysis.

In this way, on the one hand, the tissue-specific composition of two flanking sections is known, and, on the other hand, the molecular-biological characteristics of one or more sections that were located between the flanking sections in the original tissue sample are also known. The quantitative fraction, the appearance, and/or the distribution pattern of the diseased tissue or diseased cells in the section or sections analyzed by molecular-biological methods can thus be reliably determined.

If, for example, one of the flanking sections has a tumor tissue fraction of 20%, and the other flanking section has a tumor tissue fraction of 80%, the pathologist can assume that the tumor tissue fraction was in the range of 20-80% in the molecular-biologically analyzed sections, which lay between the flanking sections in situ. It may also be possible, assuming a gradient-like change in the tissue-specific composition from one section to the next, to determine or calculate the average tumor tissue fraction of the molecular-biologically analyzed sections from the tumor tissue fractions of the flanking sections. This makes it possible in these cases as well to determine a reference quantity with which, for example, the experimentally determined gene expression pattern can be corrected and then directly compared with the patterns stored in the data banks.

This is possible especially when, in addition to the flanking sections, one or more other sections that were located in situ between the molecular-biologically analyzed sections are analyzed for their tissue-specific composition. The previously described embodiment of the method of the invention should also expressly include a modification of this type.

Similar advantages apply to another preferred modification of the method of the invention, in which the divided samples (i.e., samples or portions of samples) that are sent for histological/cytological examination are selected to ensure that the one or more divided samples sent for nonmorphological analytical testing were located between these divided samples in situ.

A preferred embodiment of the method provides that, as has already been noted, the method is used for the purpose of intraoperative clinical-pathological diagnosis. The method can likewise be used for perioperative diagnostic work, i.e., shortly before or shortly after a surgical procedure.

In other preferred embodiments of the method, a method for detecting genomic DNA, cDNA, mRNA, the epigenomic methylation pattern, proteins, viral or bacterial nucleic acids, or other biomolecules is used in the nonmorphological analytical testing. Biophysical methods can also be employed in the nonmorphological analytical testing, e.g., methods for measuring the redox potential, the pH, the temperature, and the oxygen partial pressure, or for determining metabolites, such as lactate or pyruvate.

The detection of genomic DNA can uncover, for example, somatic mutations, which can be correlated with various tissue diseases. The detection of cDNA, mRNA or proteins can provide information about a disease-specific gene expression pattern. Under certain circumstances, the detection of the epigenomic methylation pattern provides indications of a disease-specific gene activation pattern. The detection of viral nucleic acids can provide information, for example, about virally provoked tissue proliferation, and the detection of bacterial nucleic acids can make it possible, for example, to diagnose bacterially provoked tissue changes. Other biomolecules, such as infectious prions, can indicate a prion-provoked tissue change. Other biomolecules may be, for example, nucleic acids or proteins of pathogenic parasites.

In another, likewise preferred, embodiment of the method of the invention, it is provided that, as part of the nonmorphological analytical testing, a quantity is determined that makes it possible to determine the fraction of diseased tissue and/or other tissue components in the tissue sample, and that the fraction thus determined is additionally used quantitatively as the basis of the evaluation of the results of the molecular-biological analysis.

An example of a quantity of this type is the degree of expression of a gene which is known to be expressed exclusively in healthy tissue but not in tumor tissue and for which the normal degree of expression is also known. The degree of expression of this gene in the molecular-biologically analyzed divided sample yields a quantity that can be used to estimate the fraction of nondiseased and diseased tissue in the sample.

Quantities of this type are sometimes referred to in the literature as “surrogate markers”. In addition to the knowledge about the tissue composition that is gained from the histological/cytological examination, the determination of the degree of expression of this gene can thus be used as the basis for evaluating the results of the molecular-biological analysis. The results of the histological/cytological examination can thus be verified by the determination of a surrogate marker.

However, quantities of this type can also include a gene expression pattern that is typical for healthy tissue or an expression ratio, which is known for a certain tumor variant, between a tumor-specific gene and a gene that is constantly expressed both in healthy tissues and in pathological tissues (“housekeeping gene”).

Moreover, the method of the invention makes it possible to detect possible surrogate markers and to validate the evidence they provide.

In another preferred modification, it is provided that a microarray or a suspension array is used as part of the nonmorphological analytical testing. Microarrays can be, for example, chips, on whose surface molecular probes are immobilized in a matrix-like arrangement (e.g., antibodies, oligonucleotides, or polypeptides), and the analytes bind to these probes due to hybridization reactions or immunological reactions and can be detected with a scanner. Microarrays are also often referred to as biochips.

Suspension arrays can be, for example, latex beads (“microbeads”), on whose surface molecular probes are immobilized (e.g., antibodies, oligonucleotides, or polypeptides), and the analytes bind to these probes due to hybridization reactions or immunological reactions and can be detected with a particle counter. Microarrays and suspension arrays are suitable, depending on the particular embodiment, both for the detection of nucleotides and for the detection of peptides or proteins.

Furthermore, in other preferred embodiments, it is provided that the biomolecules to be detected are subjected to a labeling step and/or that the nucleic acids to be detected are subjected to an amplification step. The labeling step can consist, for example, in circumscribing the mRNA molecules present in the divided sample into labeled cRNA molecules by the use of T7 RNA polymerase and Cy3-labeled ribonucleotides. Another example of a labeling step is the labeling of proteins present in the divided sample by the use of fluorescence-labeled antibodies or oligonucleotides. However, other labeling steps known from the present or future state of the art are also conceivable.

The amplification step can consist, for example, in a PCR amplification technique, an RT-PCR amplification technique, or a different amplification technique in accordance with the present or future state of the art.

In other preferred modifications, it is provided that the histological/cytological examination includes at least one staining step and/or at least one immunohistochemical step and/or in situ hybridization step. In this regard, it can be provided that several sections or tissue core samples are each subjected to different histological/cytological and/or immunohistochemical tests in order to determine a larger number of parameters. For example, one divided sample can be stained with hematoxylin and eosin to allow visualization and quantitative determination of the cell nuclei and the frequency of mitoses, while a second divided sample can be treated with a tumor-specific antibody (e.g., anti-S-100 against malignant melanomas), and a third divided sample can be treated, for example, by in situ hybridization.

It is additionally provided that a method in accordance with any of the preceding claims can be used for developing a tumor data bank, for developing individualized cancer therapies, for adjustment of a patient to an individualized cancer therapy, and/or for answering scientific questions.

A possible embodiment of the invention is described below on the basis of a nonexclusive example.

Two tumors of the same type are macroscopically evaluated and prepared in a quick section examination and then, embedded in an embedding agent, frozen in a freezing microtome on a suitable slide.

Sections 6 μm thick are then prepared. The first and the fifth sections are mounted on a slide and stained with hematoxylin-eosin according to a standard protocol. The sections are first histologically evaluated. Both are identified as carcinoma of type X. The tissue composition is then quantitatively determined for each section by means of digital image processing. The results of the morphological analysis of the hematoxylin-eosin-stained preparations provide the following picture:

TABLE 1
Histologically determined tissue composition of the two tumors.

| | Tumor Parenchyma | Tumor Stroma |
|---|---|---|
| Tumor A | 80% | 20% |
| Tumor B | 50% | 50% |

The second, third and fourth sections (each 30 μm thick) are dissolved in a reaction vessel with RNAlater to preserve the integrity of the mRNA. After total RNA extraction with a commercial kit (e.g., RNeasy by Qiagen), the poly-A RNA is selectively amplified (Wang et al., Nature Biotechnology, April 2001). One or two amplification rounds can be performed, as required. After a final labeling step with a commercial labeling kit, the amplified RNA or cDNA is prepared and can be analyzed by hybridization on a DNA microarray.

All of the mRNA molecules present in the analyzed tissue are quantitatively determined in this way. A quantitative gene expression profile is thus obtained, which can be compared with available standard gene expression profiles of various types of tissues and tumors for the purpose of more precisely defining the diagnosed tumor. Standard gene expression profiles of this type are presently being determined in various laboratories around the world for a large number of tumor and tissue types.

In the present example, the expression of the genes A to I was analyzed. The following gene expression profiles were obtained for the two analyzed tumors (values in arbitrary units):

TABLE 2
Gene expression profiles of the two tumors
determined by array experiments.

| Gene | A | B | C | D | E | F | G | H | I |
|---|---|---|---|---|---|---|---|---|---|
| Tumor A | 2.4 | 4 | 0 | 0 | 0.8 | 2.4 | 3.2 | 2.4 | 1.6 |
| Tumor B | 1 | 2.5 | 1.5 | 2 | 0 | 1.5 | 1.5 | 1 | 0.5 |

The knowledge about the tumor composition of the two tumors is now used for correcting the results of the array experiments. It is assumed here that the tissue in sections 2, 3, and 4 has a structure comparable to that of the tissue in the “marginal sections” 1 and 5.

Taking into consideration the knowledge that tumor A has a parenchymal fraction of 80% and that tumor B has a parenchymal fraction of 50%, the following standardized gene expression profiles are obtained:

TABLE 3
Standardized gene expression profiles of the two tumors.

| Gene | A | B | C | D | E | F | G | H | I |
|---|---|---|---|---|---|---|---|---|---|
| Tumor A | 3 | 5 | 0 | 0 | 1 | 3 | 4 | 3 | 2 |
| Tumor B | 2 | 5 | 3 | 4 | 0 | 3 | 3 | 2 | 1 |

The standard gene expression profiles of three subtypes of carcinoma X are known from the literature:

TABLE 4
Standard gene expression profiles of
three subtypes of carcinoma X.

| Gene | A | B | C | D | E | F | G | H | I |
|---|---|---|---|---|---|---|---|---|---|
| Subtype 1 | 2 | 5 | 3 | 4 | 0 | 3 | 3 | 2 | 1 |
| Subtype 2 | 3 | 5 | 0 | 0 | 1 | 3 | 4 | 3 | 2 |
| Subtype 3 | 2 | 4 | 2 | 1 | 3 | 5 | 3 | 2 | 3 |

A comparison of the standardized gene expression profiles with the standard gene expression profiles shows that tumor A is subtype 2 of carcinoma X, while tumor B is subtype 1 of carcinoma X.

The differentiation of the two tumors is possible only by the array analysis and the subsequent standardization of the gene expression profiles. The histological examination allows only identification of the tumor as carcinoma X but not differentiation into the various subtypes described above.

1-23. (canceled)

24. A method of analyzing a patient tissue sample to determine a fraction of diseased tissue while essentially preserving at least one of genomic property, proteomic property, epigenomic property and biophysical property of the tissue sample, the method comprising the steps of:
preparing sections from the tissue sample;
subjecting at least one of the prepared sections to a histological/cytological examination; and
subjecting at least another one of the prepared sections to a non-morphological analytical testing,
wherein in the histological/cytological examination, at least one of a quantitative fraction of diseased tissue or cells and another morphological aspect of the at least one of the prepared sections is determined by an image processing system, and
wherein the determined at least one of a quantitative fraction of diseased tissue or cells and another morphological aspect is used as a reference quantity on which evaluation of a result of the non-morphological analytical testing is based.

25. A method of analyzing a patent tissue sample to determine a fraction of diseased tissue while essentially preserving at least one of genomic property, proteomic property, epigenomic property and biophysical property of the tissue sample, the method comprising the steps of:
taking a sample from the tissue sample;
subjecting at least one portion of the sample to a histological/cytological examination; and
subjecting at least another portion of the sample to a non-morphological analytical testing,
wherein in the histological/cytological examination, at least one of a quantitative fraction of diseased tissue or cells and another morphological aspect of the at least one portion of the sample is determined by an image processing system, and
wherein the determined at least one of a quantitative fraction of diseased tissue or cells and another morphological aspect is used as a reference quantity on which evaluation of a result of the non-morphological analytical testing is based.

26. The method of claim 25, wherein the sample is taken by one of a core sampler, an aspiration, and a scraper sampling technique.

27. The method of claim 24, wherein in the histological/cytological examination, at least one of appearance and distribution pattern of the diseased tissue or cells in the tissue sample is determined and used as a basis for evaluation of the result of the non-morphological analytical testing.

28. The method of claim 25, wherein in the histological/cytological examination, at least one of appearance and distribution pattern of the diseased tissue or cells in the tissue sample is determined and used as a basis for evaluation of the result of the non-morphological analytical testing.

29. The method of claim 24, wherein the sections are prepared directly from the tissue sample when the tissue sample is fresh.

30. The method of claim 25, wherein the sample is taken directly from the tissue sample when the tissue sample is fresh.

31. The method of claim 24, wherein the tissue sample is frozen before the sections are prepared.

32. The method of claim 25, wherein the tissue sample is frozen before the sample is taken.

33. The method of claim 24, wherein the tissue sample is mounted on a slide and frozen immediately after the tissue sample is removed from a patient, and the sections are prepared from the frozen tissue sample with a microtome.

34. The method of claim 33, wherein after the sections are prepared, the tissue sample is left on the slide so that further sections can be prepared from the tissue sample.

35. The method of claim 34, wherein the slide is reproducibly placed in the microtome so that the tissue sample has the same orientation relative to the microtome in repeated preparations of sections.

36. The method of claim 24, wherein two of the prepared sections are subjected to the histological/cytological examination, and the two of the prepared sections are selected so that the at least another one of the prepared sections subjected to the non-morphological analytical testing is between the two of the prepared sections in situ.

37. The method of claim 25, wherein two portions of the sample are subjected to the histological/cytological examination, and the two portions are selected so that the at least another portion of the sample subjected to the non-morphological analytical testing is located between the two portions in situ.

38. The method of claim 24, wherein said method is used for intra-operative or peri-operative clinical diagnosis or for experimental pathological analysis.

39. The method of claim 25, wherein said method is used for intra-operative or peri-operative clinical diagnosis or for experimental pathological analysis.

40. The method of claim 24, wherein the non-morphological analytical testing comprises detecting bio-molecules or determining a biophysical characteristic of the at least another one of the prepared sections, the bio-molecules including genomic DNA, cDNA, mRNA, epigenomic methylation pattern, proteins, viral or bacterial nucleic acids, infectious prions, nucleic acids or proteins of pathogenic parasites.

41. The method of claim 25, wherein the non-morphological analytical testing comprises detecting bio-molecules or determining a biophysical characteristic of the at least another portion of the sample, the bio-molecules including genomic DNA, cDNA, mRNA, epigenomic methylation pattern, proteins, viral or bacterial nucleic acids, infectious prions, nucleic acids or proteins of pathogenic parasites.

42. The method of claim 24, wherein the non-morphological analytical testing comprises determination of a quantity which makes it possible to determine at least one of a fraction of diseased tissue and a fraction of another tissue component in the tissue sample, and the at least one of a fraction of diseased tissue and a fraction of another tissue component so determined is used quantitatively as a basis of evaluation of the result of the non-morphological analytical testing.

43. The method of claim 25, wherein the non-morphological analytical testing comprises determination of a quantity which makes it possible to determine at least one of a fraction of diseased tissue and a fraction of another tissue component in the tissue sample, and the at least one of a fraction of diseased tissue and a fraction of another tissue component so determined is used quantitatively as a basis of evaluation of the result of the non-morphological analytical testing.

44. The method of claim 24, wherein one of a micro-array and a suspension array is used in the non-morphological analytical testing.

45. The method of claim 25, wherein one of a micro-array and a suspension array is used in the non-morphological analytical testing.

46. The method of claim 40, wherein the bio-molecules to be detected by the non-morphological analytical testing are subjected to a labeling step.

47. The method of claim 40, wherein the nucleic acids to be detected by the non-morphological analytical testing are subjected to an amplification step.

48. The method of claim 41, wherein the bio-molecules to be detected by the non-morphological analytical testing are subjected to a labeling step.

49. The method of claim 41, wherein the nucleic acids to be detected by the non-morphological analytical testing are subjected to an amplification step.

50. The method of claim 24, wherein the histological/cytological examination comprises at least one staining step.

51. The method of claim 25, wherein the histological/cytological examination comprises at least one staining step.

52. The method of claim 24, wherein the histological/cytological examination comprises at least one of an immunohistochemical step and an in situ hybridization step.

53. The method of claim 25, wherein the histological/cytological examination comprises at least one of an immunohistochemical step and an in situ hybridization step.

54. The method of claim 24, wherein at least two of the sections are each subjected to different histological/cytological examinations.

55. The method of claim 24, wherein said method is used to develop a tumor data bank.

56. The method of claim 25, wherein said method is used to develop a tumor data bank.

57. The method of claim 24, wherein said method is used to develop an individualized cancer therapy.

58. The method of claim 25, wherein said method is used to develop an individualized cancer therapy.

59. The method of claim 24, wherein said method is used to adjust a patient's individualized cancer therapy.

60. The method of claim 25, wherein said method is used to adjust a patient's individualized cancer therapy.
